(19) United States
(12) Patent Application Publication
(10) Pub. No.: US 2021/0179993 A1
(43) Pub. Date: Jun. 17, 2021

(54) MODULAR FLOW-THROUGH CARTRIDGE BIOREACTOR SYSTEM

(71) Applicant: THE SECANT GROUP, LLC, Telford, PA (US)

(72) Inventors: Brian GINN, Chalfont, PA (US); Peter D. GABRIELE, Frisco, TX (US); Jeremy J. HARRIS, Doylestown, PA (US); Amanda K. WEBER, Macungie, PA (US)

(21) Appl. No.: 17/125,146

(22) Filed: Dec. 17, 2020

Related U.S. Application Data
(60) Provisional application No. 62/949,086, filed on Dec. 17, 2019.

(51) Int. Cl. C12M 3/00; C12M 1/00; C12M 1/12

(52) U.S. Cl. CPC C12M 23/42; C12M 29/04; C12M 23/44; C12M 23/58; C12M 25/16

(57) ABSTRACT

A modular flow-through cartridge bioreactor system includes a plurality of modular flow-through cartridges. Each modular flow-through cartridge includes a cartridge housing with ports for through flow of a biological media and predetermined contents preloaded in the cartridge housing permitting the cartridge to perform at least one predetermined function of the bioreactor process upon through-flow of the biological media. The modular flow-through cartridge bioreactor system also includes at least one interlock connector fluidly connecting the plurality of modular flow-through cartridges by the ports. A modular flow through cartridge includes rows of porous textiles preloaded in the cartridge housing. A process includes selecting a plurality of modular flow-through cartridges to perform, in combination, a bioreactor process. The process also includes fluidly connecting the modular flow-through cartridges in a fluid sequence to form the modular flow-through cartridge bioreactor system. Flowing the biological media through the fluid sequence performs the bioreactor process.

### CROSS-REFERENCE TO RELATED APPLICATIONS

This application claims priority to and the benefit of U.S. Provisional Application No. 62/949,086 filed Dec. 17, 2019, which is hereby incorporated by reference in its entirety.

### FIELD OF THE INVENTION

The present disclosure is generally directed to bioreactor systems. More specifically, the present disclosure is directed to modular bioreactor systems using one or more flow-through cartridges.

### BACKGROUND OF THE INVENTION

Many conventional bioprocessing reactors for expansion of cells or production of biologics are large-scale systems, where any changes to a production process are associated with high resource costs, are labor intensive, and have extensive time requirements to test and monitor potential process improvements in biologic or cell mass yield.

There are substantial limitations in conventional cell culture. Because biological production processes conventionally often take place in large-scale bioreactors, improvements to processes are hard to justify due to high development and implementation costs. A new treatment for cells or substrate currently requires small-scale testing in conditions that do not match commercial production process conditions. For example, a new type of microcarrier may initially be tested on a small scale, such as, for example, in small 150-mL spinner flasks, but their end implementation would be in larger scale systems, such as, for example, 50-L bioreactors, with completely different fluid dynamics and reactor configurations, leading to inefficient process transfer to larger scale, wasting a lot of time and money and driving up production costs.

Other bioprocessing systems, such as point-of-care systems, including allogeneic or autologous cell therapies, are run on a small scale, and may be open or closed systems that are difficult or costly to automate, customize, or adapt for single use.

### BRIEF DESCRIPTION OF THE INVENTION

It would be desirable to have a scalable modular cartridge system that is customizable to enable a bioreactor process based on the interconnectivity and function of individual cartridges.

In an embodiment, a modular flow-through cartridge bioreactor system includes a plurality of modular flow-through cartridges. Each modular flow-through cartridge includes a cartridge housing having a first port and a second port for through flow of a biological media and predetermined contents preloaded in the cartridge housing permitting the cartridge to perform at least one predetermined function of the bioreactor process upon through-flow of the biological media. The modular flow-through cartridge bioreactor system also includes at least one interlock connector fluidly connecting the plurality of modular flow-through cartridges by the first ports and the second ports.

In another embodiment, a modular flow through cartridge includes a cartridge housing having a first port and a second port for through flow of a biological media and rows of porous textiles preloaded in the cartridge housing. The first port and the second port are modularly configured to fluidly couple to a first port and a second port of a second modular flow through cartridge.

In yet another embodiment, a process of constructing a modular flow-through cartridge bioreactor system includes selecting a plurality of modular flow-through cartridges to perform, in combination, a bioreactor process. Each modular flow-through cartridge includes a cartridge housing having a first port and a second port for through flow of a biological media and predetermined contents preloaded in the cartridge housing permitting the cartridge to perform at least one predetermined function of the bioreactor process upon through-flow of the biological media. The process also includes fluidly connecting the plurality of modular flow-through cartridges by the first ports and the second ports in a fluid sequence to form the modular flow-through cartridge bioreactor system. Flowing the biological media through the fluid sequence performs the bioreactor process.

Various features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 shows a schematic side view of a two-cartridge system in an embodiment of the present disclosure.

FIG. 2 shows a schematic side view of a cartridge with flow parallel to textile layers in an embodiment of the present disclosure.

FIG. 3 shows a schematic series configuration of cartridges in an embodiment of the present disclosure.

FIG. 4 shows a schematic parallel configuration of cartridges in an embodiment of the present disclosure.

FIG. 5 schematically shows a separation cartridge system in an embodiment of the present disclosure.

FIG. 6 schematically shows a cartridge with microparticles in an embodiment of the present disclosure.

FIG. 7A shows the flow cytometry data of all events of a mixture of Jurkat and Chinese hamster ovary (CHO) cells prior to exposure to microspheres.

FIG. 7B shows the flow cytometry data of cells of the mixture of FIG. 7A.

FIG. 7C shows the flow cytometry data of all events of a supernatant of the mixture of Jurkat and CHO cells after exposure to microspheres.

FIG. 7D shows the flow cytometry data of cells of the mixture of FIG. 7C.

Wherever possible, the same reference numbers will be used throughout the drawings to represent the same parts.

### DETAILED DESCRIPTION OF THE INVENTION

Exemplary embodiments permit the construction of a modular flow through cartridge system. In exemplary embodiments, the system is a modular flow through cartridge bioreactor system.

The modular flow-through cartridge bioreactor system is constructed of multiple flow-through cartridges, each cartridge including a cartridge housing preloaded with contents to permit it to perform at least one predetermined function of the bioreactor process. Each cartridge housing includes at least a first port and a second port, one serving as an inlet port and the other as an outlet port for flow-through functionality. In some embodiments, each cartridge performs one predetermined function of an overall process performed by the modular flow through cartridge bioreactor system. Each cartridge is preferably single use, sterilized prior to the initial use, and disposed of after the initial use, such that no sterilization or cleaning is required following use.

In exemplary embodiments, the modularity includes that each port of each cartridge is attachable to each port of each other cartridge in the modular flow through cartridge system. The modular sequence design of the cartridges enables unique bioreactor configurations, such as, for example, one where an upstream cartridge contains a feeder cell line that is physically separated from a downstream cell type that receives beneficial cytokines produced by the feeder cells.

By having a scalable, modular system, exemplary embodiments drive down the development costs of improving the production efficiency of biologics. The lowered development costs and modular nature of the cartridge also act as an enabler for producing patient-specific vaccines, therapeutics, cell therapies, and gene therapies, which would otherwise be too costly to produce in a small-scale custom batch process. Similarly, lowered development costs and customizability of the cartridge system may help biopharma companies pursue biologics markets for rarer afflictions that currently do not have enough potential market size to be seen as worth resource investment by biopharma.

It will further be appreciated that the modularity of the cartridges gives additional flexibility in the manner in which both the cartridges and the overall system are arranged, as well as the manner in which the cartridges are manipulated, and their contents recovered.

The cartridges may be arranged into a 2D planar footprint pattern, such as, for example, a 3×3×1 grid in x-y-z space, or into a 3D volumetric footprint pattern, such as, for example, a 3×3×3 grid in x-y-z space. In some embodiments, the cartridges are arranged to generate cellular circuits.

The preloaded contents of a cartridge and the cartridge's position in a modular flow through cartridge bioreactor system define the function the cartridge performs. In exemplary embodiments, a flow-through cartridge contains at least one porous textile. Distinct cartridges containing textiles modified to collect particular biologics can be used in sequence to initially separate one or more different produced biological factors in a single flow pathway.

Porous textiles provide a high surface area for adherent cell culture in a given volumetric footprint, enabling cell expansion to densities within a small cartridge that directly correlate to the cell concentrations used for commercial-scale biologic production.

Further, the porosity of the textile can be tuned for a particular application to be, for example, either larger for perfusion with adherent cells or reduced to smaller values to enable culture of suspension cells within a cartridge.

In some embodiments, the textiles are anchored within the cartridge and stacked into multiple layers with spacing between the textile layers. A series of porous textiles are placed within a sealed cartridge for use in the culture of adherent cells. One or more such cartridges are then placed in sequence so that media is perfused through the porous textile structure containing adherent cells.

The porous textiles may include any of woven, non-woven, knit, braided structures, or a combination thereof, as well as electrospun meshes which may be used in place of, or in combination with other forms of textile inside a cartridge. The textile materials are typically composed of synthetic polymers such as, but not limited to: poly(lactic-co-glycolic acid) (PLGA), poly(lactic acid) (PLA), polyglycolide (PGA), polycaprolactone (PCL), poly(ethylene terephthalate) (PET), poly(vinylidene fluoride) (PVDF), polyethersulfone (PES), polypropylene (PP), and blends thereof, by way of example only. In some embodiments, the materials are composed of biologically derived polymers, which may include, but are not limited to, collagen, fibrin, alginate, hyaluronic acid, other polysaccharides, silk, cellulose, gelatin, and blends thereof. In still other embodiments, the materials are composed of a conductive polymer so that an electric potential can be applied to polarize cells.

For a given cartridge size, the number of textiles and their spacing within the cartridge may be varied to tune for maximum cell carrying capacity per unit for a particular application.

In some embodiments, a pocket weave structure is used for the porous textiles. For example, a pocket weave structure placed along the inner wall of the cartridge can capture or contain cells within the textile pocket. A pocket weave structure placed throughout the core of the cartridge can also be used to capture or contain cells. A pocket weave structure within a cartridge can also be used to contain feeder material that is released into the culture media over time. That feeder material within the pocket weave may be composed of cell metabolites, amino acids, an active pharmaceutical ingredient release device, pH balancing reagents, a cell antagonist designed to negatively stimulate cells or a combination of these and/or other feeder materials.

In some embodiments, a cartridge contains multilayered textile structures that have an intentionally varied porosity per layer.

The porous textiles within a cartridge may be coated. In some embodiments, textiles are coated with cell integrin binding motifs such as those containing amino acid sequences of: arginine-glycine-aspartic acid (RGD), isoleucine-lysine-valine-alanine-valine (IKVAV), tyrosine-isoleucine-glycine-serine-arginine (YIGSR), and others. In other embodiments, the cartridge textiles are coated with a poly(glycerol sebacate) (PGS) based composition, which include coating with a PGS composition containing amino acids, an active pharmaceutical ingredient (API), other soluble cell cytokines, or combinations thereof. The PGS-based composition may include any form of PGS polymer or copolymer, such as poly(glycerol sebacate) urethane (PGSU) or a poly(glycerol sebacate) acrylate (PGSA), for example. In some embodiments, the PGS has signaling proteins tethered to the PGS surface.

In addition or alternatively to a porous textile, each cartridge may have other preloaded contents to aid in the function provided by the cartridge. A particular cartridge may have one or more particular features or contents depending on the particular function the particular cartridge is intended to provide. In some embodiments, a cartridge contains microsphere cell carriers, non-spherical cell carriers, or both. A cartridge may also contain microspheres modified to capture biologics and/or cells and/or to release metabolites, cytokines, proteins, biologics, cells, and/or API.

In some embodiments, inlet and/or outlet regions of cartridges contain a porous filter of a porous filter material. Appropriate porous filters may include, but are not limited to, particulates, such as beads, microparticles, microspheres, macrospheres, nanospheres, nanoparticles, or irregularly-shaped flour-like materials; porous woven or non-woven textiles; sponge-like materials with interconnected pores; solid materials with integrated flow through channels; or hydrogel materials. Appropriate porous filter materials may include, but are not limited to, synthetic polymers, such as, for example, polytetrafluoroethylene (PTFE), PVDF, PET, PLGA, poly(methyl methacrylate) (PMMA), PLA, PGA, PCL, polystyrenes, polyethylenes, or PGS; biologically derived materials such as collagen, cellulose, or alginate; or porous metals. It will be appreciated that the selection for the material of construction for the filter materials may be selected for adherence or non-adherence of cells, which may depend on the ultimate application for which the cartridges are being employed processing is being used.

The pore size of the porous filter may be selected based on the size of the materials to be retained or passed through the porous filter. For example, the pore size may be selected to separate microspheres from trypsinized cells, cell aggregates of different sizes from each other, microspheres of different sized from each other, microspheres from biologics, cells from biologics. Appropriate pore sizes for separation of differing microspheres or cell aggregates may include, but are not limited to, about 50 μm to about 150 μm, about 150 μm to about 250 μm, about 250 μm to about 350 μm, about 350 μm to about 450 μm, about 450 μm to about 550 μm, about 550 μm to about 750 μm, about 750 μm to about 1000 μm, about 1000 μm to about 1500 μm, about 1500 μm to about 2000 μm, or ranges >2000 μm in diameter, or any value, range, or sub-range therebetween. Appropriate pore sizes for separation of cells from aggregates or microspheres may include, but are not limited to, about 10 μm to about 20 μm, about 10 μm to about 30 μm, about 10 μm to about 40 μm, about 10 μm to about 50 μm, about 10 μm to about 100 μm in diameter, or any value, range, or sub-range therebetween. Appropriate pore sizes for separation of biologics from cells, aggregates, or microspheres may include, but are not limited to, about 1 nm to about 100 nm, about 1 nm to about 250 nm, about 1 nm to about 500 nm, about 1 nm to about 1000 nm, about 1 nm to about 5000 nm, about 1 nm to about 10,000 nm, about 1 nm to about 100,000 nm diameter, or any value, range, or sub-range therebetween.

In some embodiments, a cartridge contains free-floating textile disks, which may be coated with PGS.

In some embodiments, a cartridge contains textiles tethered with a functionality that captures cell waste products or inhibitory cytokines during media recirculation through the cell circuit. In some embodiments, a cartridge contains signaling molecules to polarize macrophages towards either M0, M1, or M2 phenotypes.

In some embodiments, a cartridge contains soluble PGS molecules in the culture media to act as an antifreeze agent within the cartridge to reduce ice crystal formation during subsequent freezing and storage or shipment of cell cartridges. Conventional cell antifreeze products are typically damaging to cells if they are not rapidly removed upon thawing of cells, but PGS does not have to be removed from media after thawing because of its breakdown components.

In some embodiments, a cartridge contains a porous fixed bed scaffolding material.

In some embodiments, a cartridge contains degradable textile layers on which cells are grown. The degradable textile layers are secured in discrete removable disks that may be individually removed from the cartridge and implanted into a patient.

In some embodiments, a cartridge contains textile scaffolding coated with biodegradable circuitry to determine changes in cell coverage on textile based on changes in conductivity. In some embodiments, the biodegradable circuitry is composed of PGS.

In some embodiments, a cartridge contains conductive textiles, which may serve as sensing elements.

In some embodiments, a cartridge contains a piezoelectric textile, which may serve as a sensing element.

In some embodiments, a cartridge contains conductive textiles as a priming component for cell and tissue types that respond to electrical stimuli such as, for example, nerves, muscle cells, or cardiac cells.

In some embodiments, a cartridge contains multilayered textile structures with multiple textile material compositions.

In some embodiments, a cartridge contains microparticles, which may act as an adherent cell scaffold, a cell or biologic sequestering matrix, or a controlled release matrix. In some embodiments, the microparticles are microspheres, microbeads, irregularly-shaped flour-like particles, or combinations thereof.

In some embodiments, a cartridge is labeled with a scannable code, such as, for example, a barcode, a quick response (QR) code, or a radio-frequency identification (RFID) code. The scannable code may identify the cartridge type or cartridge contents, such as in automated systems.

It will be appreciated that cartridges may also be constructed to contain individual sensors to monitor cartridge-specific microenvironments.

In some embodiments, a cartridge contains a medical device for testing, such as, for example, a vascular graft.

In some embodiments, a cartridge acts as a bioreactor for the creation of organ structures.

In some embodiments, a cartridge is loaded with an organ-templated scaffold that allows for cell colonization and growth to create an implantable device to replace diseased or damaged tissue.

Once the cartridges are selected and connected in a predetermined arrangement to form a predetermined modular flow-through cartridge bioreactor system, flow of media then commences through an interlock between a first cartridge to one or more second cartridges downstream containing additional cell types or modified textile surfaces to capture biologics produced by upstream cells.

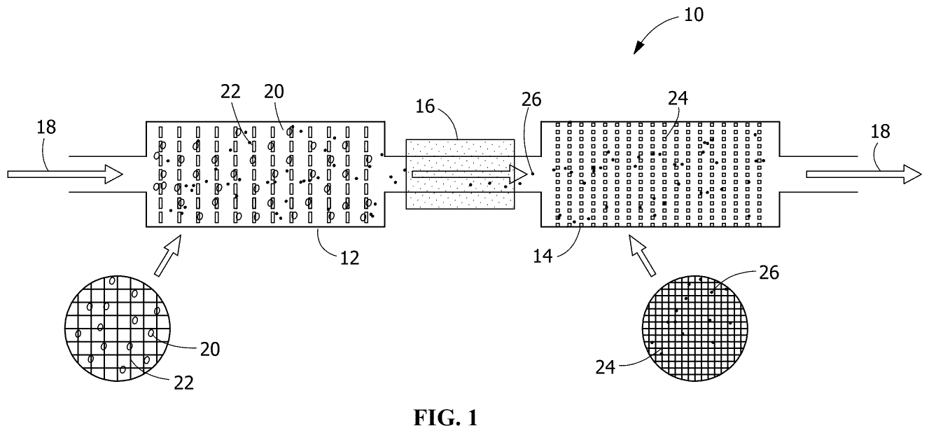

Referring to FIG. 1, a two-cartridge system 10 includes an upstream cartridge 12 that is a textile cell culture cartridge and a downstream cartridge 14 that is a biologics collection cartridge. The upstream cartridge 12 is fluidly connected to the downstream cartridge 14 by an interlock connector 16 to permit flow of media 18 into the upstream cartridge 12, through the interlock connector 16, and into the downstream cartridge 14. The upstream cartridge 12 contains cells 20 adherent upon rows of a cell culture textile 22, where the cell culture textile 22 is a porous textile weave. The downstream cartridge 14 contains rows of a biologic collection textile 24. The textiles 22, 24 are oriented perpendicular to the general direction of flow of the media 18. The adherent cells 20 produce biologics 26, which are transported by the media 18 and are collected in the downstream cartridge 14 on the biologic collection textile 24. The biologic collection textile 24 has a high surface area textile surface modified with antibodies to scavenge the target biologic 26. Once saturated with biologic 26, the downstream cartridge is replaced with a new biologic collection cartridge and the captured biologic 26 is retrieved from the downstream cartridge 14 that was removed and is then purified.

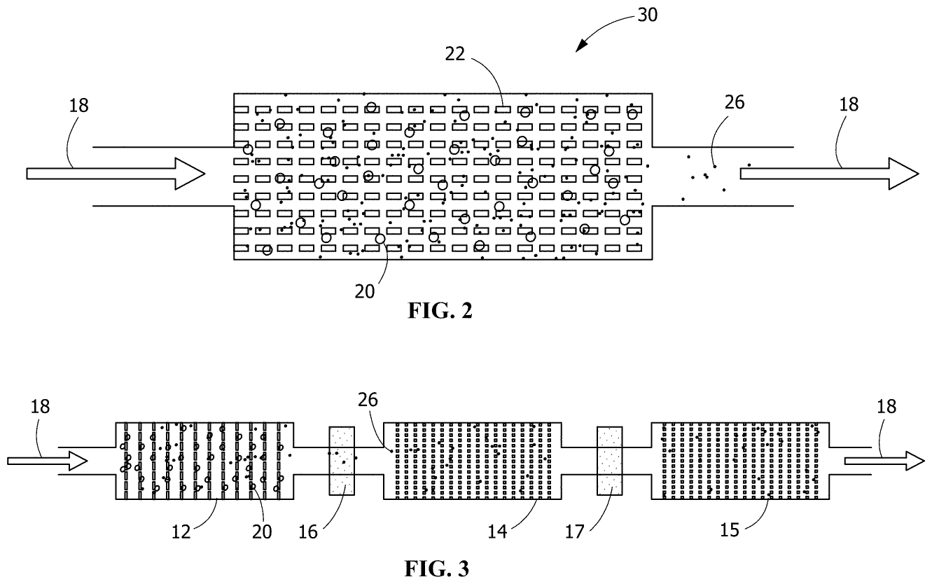

In some embodiments, the textile layers are oriented in the cartridge such that media flow is tangential to the textile surface rather than orthogonally perfusive. Referring to FIG. 2, the tangential flow cartridge 30 is a textile cell culture cartridge. The tangential flow cartridge 30 contains cells 20 adherent upon rows of a cell culture textile 22. The cell culture textiles 22 are oriented parallel to the general direction of flow of the media 18. The adherent cells 20 produce biologics 26, which are transported by the media 18 out of the tangential flow cartridge 30.

When multiple cartridges are employed, a series type and/or a parallel type cell circuit may be employed, including variations that include some combination of the two circuit types.

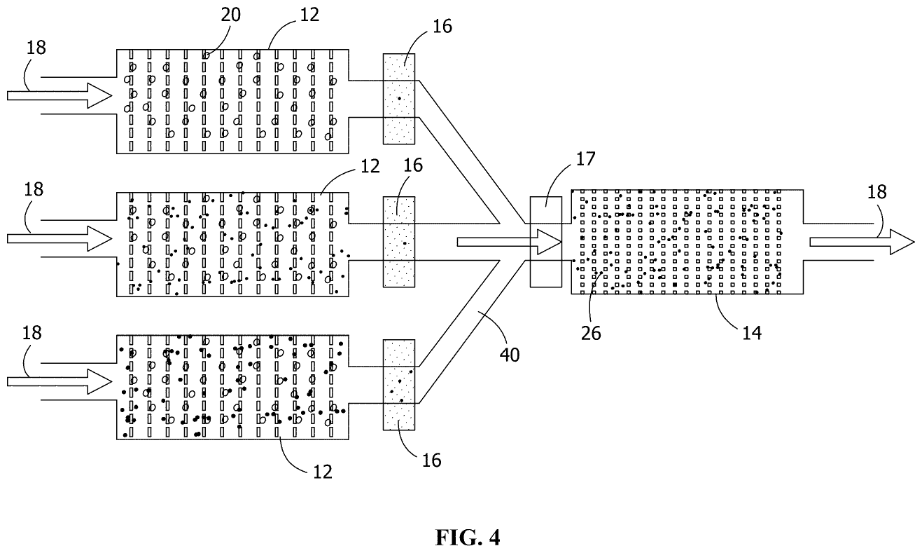

Referring to FIG. 3, a cell culture cartridge 12 is arranged upstream in series with a first biologic collection cartridge 14 and a second biologic collection cartridge 15. The cell culture cartridge 12 is fluidly connected to the first biologic collection cartridge 14 by a first interlock connector 16, and the first biologic collection cartridge 14 is fluidly connected to the second biologic collection cartridge 15 by a second interlock connector 17 to permit flow of media 18 into the cell culture cartridge 12, through the first interlock connector 16, into the first biologic collection cartridge 14, through the second interlock connector 17, and into the second biologic collection cartridge 15. The first biologic collection cartridge 14 and the second biologic collection cartridge 15 may collect the same biologic or different biologics.

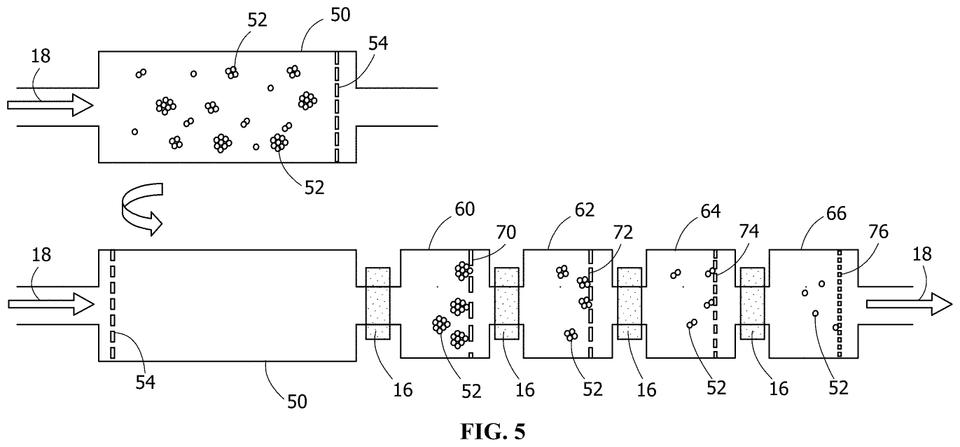

Referring to FIG. 4, a first cell culture cartridge 12, a second cell culture cartridge 12, and a third cell culture cartridge 12 are arranged in parallel upstream of a biologic collection cartridge 14. The cell culture cartridges 12 are fluidly connected to a combination joint 40 by three first interlock connectors 16. The combination joint 40 combines the flows of media 18 and is fluidly connected to a biologic collection cartridge 14 by a second interlock connector 17. The flows of media 18 travel into the cell culture cartridges 12, through the first interlock connectors 16, into the combination joint 40, through the second interlock connector 17, and into the biologic collection cartridge 14. The cell culture cartridges 12 may all be the same or may be different.

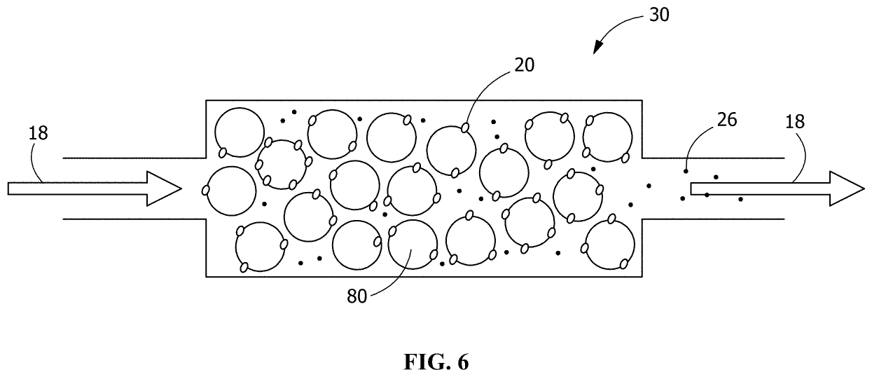

In other embodiments, cartridges may be selected and arranged to separate based on size. Referring to FIG. 5, a first cartridge 50 contains cell aggregates 52 of different sizes. The cell aggregates 52 may be initially washed with a flow of media 18, with a downstream first filter 54 retaining the cell aggregates 52 in the first cartridge 50. The first cartridge 50 is then connected in the reverse flow orientation to a series of separation cartridges 60, 62, 64, 66, each with a textile filter 70, 72, 74, 76 with a decreasing textile pore size, respectively, relative to the previous upstream separation cartridge, to collect and separate cell aggregates 52, based on aggregate diameter, upon flow of media 18.

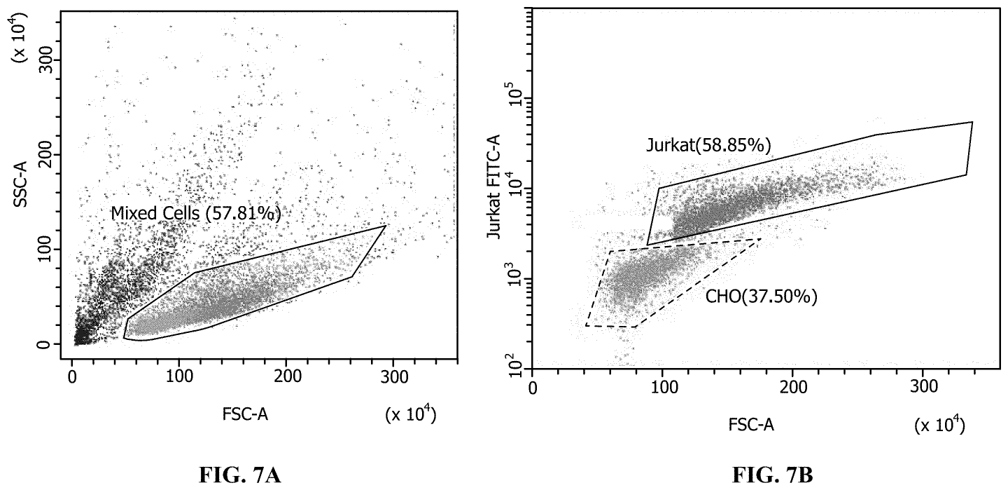

Referring to FIG. 6, a cartridge 30 is preloaded with microparticles 80. The cartridge 30 also contains cells 20 adherent upon the microparticles 80. The adherent cells 20 produce biologics 26, which are transported by the flow of media 18 out of the cartridge 30.

With a unified modular cartridge system in place, it becomes much easier to translate academic and clinical discoveries to commercial production for widespread deployment, because the small-scale cartridge configurations may be directly scaled to larger scale versions of the cartridges, which may be either in hard-plastic containment or soft-plastic bags.

In some embodiments, the cartridge has a hard outer shell, typically of plastic, to ease automation, although soft plastic containment may alternatively be used. Exemplary materials for the outer shell may include, but are not limited to, polycarbonates (PC), polystyrene (PS), acrylonitrile butadiene styrene copolymers (ABS), polyurethanes (PU), high or low density polyethylene (LDPE, HDPE), polyvinyl chloride (PVC), PVDF, polysulfones (PSU), polyetheretherketone (PEEK), urethane thermoplastic elastomers (TPU), PET, polyamides, or blends thereof. In some embodiments, the cartridges may be constructed with a hard shell composed of metals such as stainless steel or of ceramic. In other embodiments, cartridges have a soft shell composed of a polymer, which may, but is not limited to, a plasticized PVC, ethylene vinyl acetate (EVA), polyethylene copolymers (PE), polypropylene (PP), polystyrene (PS), blends, or laminates thereof.

The modular cartridge bioreactor system can be constructed to interface with existing perfusion systems and controller units. Cartridge units may be connected through a clamping mechanism and can be compatible with sizes of tubing and connectors used in existing conventional systems.

In some embodiments, the cartridges and cell circuit is contained within a modular bioisolation system. Additionally, the cartridges may include an interlock region compatible with luer connectors having a predetermined size, including, but not limited to, 1/16″ (1.6 mm), ⅛″ (3.2 mm), ¼″ (6.4 mm), or larger and/or which make use of interlocks between cartridges that contain a quick-release mechanism. In some embodiments, the inlets and outlets of the cartridge are compatible with a luer lock system and valves can be placed into the interlock region between cartridges for user needs, such as, for example, diverting flow, preventing backflow with a check valve, or making sensor measurements. The connector region between cartridges may contain bypass flow pathways and flow redirects to enable continuous operation of the cell circuit during exchange of cartridges.

Cartridges are arranged into a cellular circuit and media is perfused through the system. In some embodiments, the perfusion is provided by a pump for circulation of nutrients and produced biologics, by gravity with the cartridges being arranged vertically, by a bioelectric current such as provided by a conductive polymer, by a pulling negative pressure, or combinations thereof.

A central controller may tune the media properties, including, but not limited to, pH, metabolite levels, measuring the amount of product, or clearance of waste. The cell circuit may be operated in either a closed loop system, where media is recirculated, or in an open loop system, where media is not recirculated and is instead fed directly into a downstream collector. Manipulation among cell circuits may be carried out manually or by an automated robotic system.

The cartridge bioreactor system allows rapid, small-scale testing of production process changes by utilizing a smaller number of cells on a perfusable porous textile mesh that is connected to other cartridges in a modular system, providing the user with a high degree of flexibility for testing processing parameters and collection of biologics produced. Small-scale systems can be used for testing and then directly translated to larger scale systems containing the same perfusion dynamics and method of biologic collection.

Thus, exemplary embodiments effectively provide a modular mini-bioreactor system that fits, for example, in a 37° C. incubator and that is more easily translatable to larger bioreactor systems than current technologies based on microfluidic systems, such as lab-on-a-chip designs. The modular nature of exemplary embodiments greatly improves customizability for process changes at reduced cost, by providing the user with a customizable cell circuit. Due to the modular nature of the cartridges, multiple bioprocessing paradigms may be tested using the same basic cartridge design, such as, for example, perfusion bioreactors, fixed bed bioreactors, suspended carrier bioreactors, adherent cells, suspension cells, and roller bottles within a cell circuit.

Unlike conventional systems, the modular nature of the cartridge configuration in accordance with exemplary embodiments allows for adjusting downstream processing, such as, for example, trypsinizing upstream cells and collecting them in a downstream cartridge, with the cartridges based on function or experiment, such as, for example, growth, biologic collection, cell capture, or removal. Thus, exemplary embodiments allow for cell culture and biologic production via a modular building block set by promoting adaptability of system modules, allowing the end-user to put together different cartridges in custom configurations. This type of functionality is particularly useful for experimentation, such as, for example, at the academic or bench-top level.

The cartridge-based system of exemplary embodiments is highly compatible with automated systems associated with large-scale cell and biologic production, enabling the cartridges to be used in a wide variety of scenarios ranging from academic to startup to large-scale production.

Among the advantages of the cartridges are that individual cartridge construction and the particular arrangement of textiles and/or microparticles within them can be engineered for a variety of functions. Appropriate broad categories of functions that may be performed by an individual cartridge may include, but are not limited to, upstream processing, downstream processing, cell expansion, containment of cell carriers, such as, for example, disks, microcarriers, or fibers, biologics collection, cell collection, therapeutic delivery, metabolite sensing, nucleic acid collection, device testing, sensor cells, cellular cryostorage, cell therapy, therapeutic testing, biologics selection, or biologics purification.

In some exemplary embodiments, these and other functions are achieved by construction of the cartridges. The porous textiles may be adherent or non-adherent. For example, in some embodiments, a large pore textile with high surface area is used for culture of adherent cells in a cartridge, while a non-adherent small pore textile is used to contain a suspension of cells or cell aggregates within a cartridge. Antibodies may be tethered to the surface of a textile and/or microparticles within a downstream cartridge to provide for positive or negative selection as the media passes through. For example, these antibodies may capture a certain cell type or scavenge produced biologics.

The cartridges may be manipulated for cell recovery in a variety of ways. In some embodiments, cartridges containing cells are physically exchanged out of the circuit and replaced by fresh cell cartridges as proliferation increases to avoid entrapment of produced biologics by cell cartridges. The cartridges may be rotated along the long cartridge axis to dislodge cell aggregates. In some embodiments, cartridges containing cells are removed from the primary cell circuit and placed in a secondary cell circuit in a reversed configuration (switch orientation of inlet and outlet) to remove cells under reverse flow. In some embodiments, sonication is used in conjunction with trypsinization to detach adherent cells from scaffolding within a cartridge. In other embodiments, downstream cartridges are designed as chromatography columns to separate and purify biologics.

In some embodiments, cartridges are placed onto a roller system and partially filled with media to mimic traditional roller bottle culture at various scales. For example, cartridges containing microspheres of sufficient density to rapidly settle are placed on a roller system for a tumble-based culture system or cartridges containing microspheres of densities near the culture media are placed on a roller system for a suspension culture system.

In exemplary embodiments, the modular flow-through cartridge bioreactor system is designed to provide features that simulate in vivo conditions of the contained cells. Appropriate simulating features may include, but are not limited to, extracellular matrix materials, biosignaling molecules, cell adhesion promotors, scaffolding, pulsatile flow, electrical stimulation, electromagnetic radiation, vibrations, or combinations thereof.

Cartridge product data, which may include, but is not limited to, scaffold content, sensor data, cell type and source, storage conditions, shipping conditions, product expiration date, manufacturing date, or sensor data, may be stored electronically such as in a database or on a blockchain and may also be embedded in a QR code that is affixed to the cartridge at the time it is removed from the cell circuit.

Exemplary embodiments provide for bioreactors for providing cell therapy, which may include cell selection, cell activation, cell transfection, and/or cell transduction, cell culture, and biologics production ranging from academic lab bench-scale setting to large-scale automated commercial production. In some embodiments, the cartridge-based system is directly scalable so that the cells are exposed to the same conditions across scales. For example, a small benchtop system may have a cartridge volume of 20 mL for testing process changes. Once changes are validated, the process may be directly transferred to larger scale cartridges with greater volume, such as 2 L or higher, while maintaining the same bioreactor features, including, but not limited to, aspect ratio or media perfusion dynamics. The flexibility of the cartridge bioreactor system in accordance with exemplary embodiments may be used to drive new innovations as, for example, a small-scale operation such as start-ups or academic research institutions can develop processes that can then be directly applied at higher production scale by the small-scale operation or a company that acquires their technologies, for more efficient point of care or custom patient solutions or any variety of other applications that realize an advantage from customizability.

Exemplary embodiments provide one or more biologics for an immunotherapy, such as, for example, generation and collection of chimeric antigen receptor (CAR) T cells. In such embodiments, cartridges perform different steps of the generation and collection of CAR-T cells, such as, for example, selection, activation, transfection, or transduction. In some embodiments, these functions are performed or provided by functionalized polymeric microparticles, polymeric nanoparticles, and/or textile structures. Catch-and-release cartridges may provide transfer and separation of functionalized polymeric microbeads based on size and collection of generated CAR-T cells. In some embodiments, the polymeric microparticles are PGS-based. In some embodiments, the textile structures are coated with PGS.

Exemplary embodiments provide cellular mimetics based on multifunctionalization of polymeric beads. In such embodiments, cartridges perform different steps of the chemical modification and separation of polymeric microparticles based on size and/or surface functionality of the microbeads. In some embodiments, the polymeric microparticles are PGS-based.

Although the invention has mainly been described with respect to biological systems such as bioreactors, it will be appreciated that the principles of the invention may be applied for use in other applications including, for example, water filtration systems, particulate sieving, chemical reactors, or metallurgy.

### Example

The invention is further described in the context of the following example, which is presented by way of illustration, not of limitation.

Cell separation was demonstrated with 212-μm to 300-μm anti-cluster of differentiation 4 (anti-CD4) PGSU microspheres with a mixture of Jurkat and CHO cells. Jurkat cells possess the CD4 protein in their cell membrane, whereas CHO cells do not. Jurkat cells therefore selectively bind to PGSU microspheres that have CD4 antibodies attached to the surface.

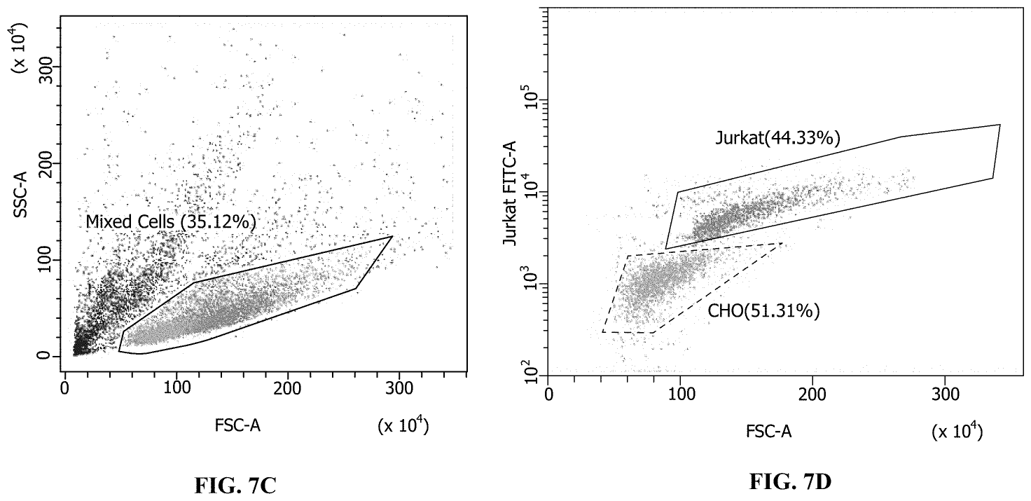

An approximately equal proportion of Jurkat and CHO cells were mixed together and analyzed before and after exposure to anti-CD4 PGSU microspheres. Jurkat cells were labeled in a 1 μM solution of the dye calcein AM, typically used as a fluorescent live cell stain, for 30 minutes at 37° C. Once stained, Jurkat cells were washed two times with Hank's Buffered Saline Solution (HBSS) for 5 min per wash. The fluorescently labeled Jurkat cells were diluted to a concentration of about 1 million cells/mL. CHO cells were washed twice with HBSS for 5 minutes each with no fluorescent staining steps and then diluted to a concentration of about 1 million cells/mL. Equal volumes of the labeled Jurkat cells were mixed with the unlabeled CHO cells to yield a final mixed concentration of about 500,000 cells of each type per mL. FIG. 7A shows the flow cytometry data of all event before further processing of the cell mixture. FIG. 7B shows the flow cytometry data of only the cells before further processing of the cell mixture. The Jurkat cells are in the upper box, and the CHO cells are in the lower box.

To perform the Jurkat cell selection, 500 μL of the Jurkat/CHO cell mixture was added to about 100 μL of the anti-CD4 PGSU microspheres. The cells and microspheres were briefly mixed with gentle pipetting and then cells were allowed to bind to the microspheres for five minutes, with occasional gentle shaking every minute during the five-minute incubation. Following the five-minute incubation, microspheres were allowed to settle for about 45 seconds, at which point the supernatant containing cells was collected and analyzed on a flow cytometer to determine the relative cell populations. Flow cytometer data showed a reduction in the relative population of Jurkat cells to CHO cells following exposure to the anti-CD4 PGSU microspheres indicating that they were preferentially selected out of the cell mixture. FIG. 7C shows the flow cytometry data of all event for the supernatant after exposure to the microspheres. FIG. 7D shows the flow cytometry data of the cells for the supernatant after exposure to the microspheres. The Jurkat cells are in the upper box, and the CHO cells are in the lower box. Table 1 shows the relative decrease of the Jurkat cells after separation with microspheres based on the preferential binding of the Jurkat cells to anti-CD4 PGSU microspheres.

TABLE 1
Relative amounts of Cells before and after Separation

| | Before Separation | After Separation |
|---|---|---|
| Jurkat Cell Population (%) | 55.9 | 44.3 |
| Number of Jurkat Events | 3402 | 1557 |
| CHO Cell Population (%) | 37.5 | 51.3 |
| Number of CHO Events | 2168 | 1802 |
| Mixed Cell Total Events | 5781 | 3512 |

While the invention has been described with reference to one or more exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A modular flow-through cartridge bioreactor system comprising:
a plurality of modular flow-through cartridges, each modular flow-through cartridge comprising:
a cartridge housing having a first port and a second port for through flow of a biological media; and
predetermined contents preloaded in the cartridge housing permitting the cartridge to perform at least one predetermined function of the bioreactor process upon through-flow of the biological media; and
at least one interlock connector fluidly connecting the plurality of modular flow-through cartridges by the first ports and the second ports.

2. The modular flow-through cartridge bioreactor system of claim 1, wherein the predetermined contents comprise rows of porous textiles.

3. The modular flow-through cartridge bioreactor system of claim 2, wherein the rows of porous textiles are oriented perpendicular to the flow of the biological media based on the location of the first port and the second port.

4. The modular flow-through cartridge bioreactor system of claim 2, wherein the rows of porous textiles are oriented parallel to the flow of the biological media based on the location of the first port and the second port.

5. The modular flow-through cartridge bioreactor system of claim 2, wherein the porous textiles are adherent for biological cells.

6. The modular flow-through cartridge bioreactor system of claim 2, wherein the porous textiles are modified with antibodies.

7. The modular flow-through cartridge bioreactor system of claim 1, wherein the predetermined contents comprise a porous filter.

8. The modular flow-through cartridge bioreactor system of claim 1, wherein the predetermined contents comprise polymeric microparticles or nanoparticles.

9. A modular flow through cartridge comprising:
a cartridge housing having a first port and a second port for through flow of a biological media; and
rows of porous textiles preloaded in the cartridge housing;
wherein the first port and the second port are modularly configured to fluidly couple to a first port and a second port of a second modular flow through cartridge.

10. The modular flow-through cartridge of claim 9, wherein the rows of porous textiles are oriented perpendicular to the flow of the biological media based on the location of the first port and the second port.

11. The modular flow-through cartridge of claim 9, wherein the rows of porous textiles are oriented parallel to the flow of the biological media based on the location of the first port and the second port.

12. The modular flow-through cartridge of claim 8, wherein the porous textiles are adherent for biological cells.

13. The modular flow-through cartridge of claim 9, wherein the porous textiles are modified with antibodies to scavenge a biologic.

14. The modular flow-through cartridge of claim 9 further comprising a porous filter at the first port.

15. The modular flow-through cartridge bioreactor system of claim 9 further comprising a porous filter preloaded in the cartridge housing.

16. A process of constructing a modular flow-through cartridge bioreactor system, the process comprising:
selecting a plurality of modular flow-through cartridges to perform, in combination, a bioreactor process, each modular flow-through cartridge comprising a cartridge housing having a first port and a second port for through flow of a biological media and predetermined contents preloaded in the cartridge housing permitting the cartridge to perform at least one predetermined function of the bioreactor process upon through-flow of the biological media; and
fluidly connecting the plurality of modular flow-through cartridges by the first ports and the second ports in a fluid sequence to form the modular flow-through cartridge bioreactor system;
wherein flowing the biological media through the fluid sequence performs the bioreactor process.

17. The process of claim 16, wherein the at least one predetermined function is selected from the group consisting of upstream processing, downstream processing, cell expansion, containment of cell carriers, biologics collection, cell collection, therapeutic delivery, metabolite sensing, nucleic acid collection, device testing, sensor cells, cellular cryostorage, cell therapy, therapeutic testing, biologics selection, biologics purification, and combinations thereof.

18. The process of claim 16, wherein the fluidly connecting comprises fluidly connecting the plurality of modular flow-through cartridges in series.

19. The process of claim 16, wherein the fluidly connecting comprises fluidly connecting at least two of the plurality of modular flow-through cartridges in parallel.
